(19) United States
(12) Patent Application Publication
(10) Pub. No.: US 2004/0006097 A1
(43) Pub. Date: Jan. 8, 2004

(54) (E)-7(4-Fluorophenyl)-6isopropyl2-mesylaminopyrimidin-5-y)-(3r,5s)-dihydroxyhept-6-enoic acid.

(76) Inventors: Steven James Hill, Macclesfield (GB); Eva Maria Lenz, Macclesfied (GB); Paul John Phillips, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 10/258,065

(22) PCT Filed: May 4, 2001
(86) PCT No.: PCT/GB01/01979

(30) Foreign Application Priority Data
May 10, 2000 (GB) 0011163.3

(51) Int. Cl. A61K 31/505; C07D 239/02

(52) U.S. Cl. 514/275; 544/332

(57) ABSTRACT

The present invention relates to the compound of formula (I): or a pharmaceutically acceptable salt or an in vivo hydrolysable ester thereof. This compound of formula (I) is conveniently called: (E)-7[4-(4-fluorophenyl)-6-isopropyl-2-mesylaminopyrimidin-5-yl]-(3R,5S)-dihydroxyhept-6-enoic acid.

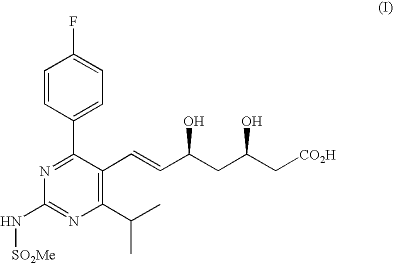

The present invention relates to a pyrimidyldihydroxyheptenoic acid derivative which is an inhibitor of 3-hydroxy-3-methylglutaryl coenzyme A (HMG-CoA) reductase, its salts, processes for its preparation and its use in medicaments.

The first generation of drags for the treatment of atherosclerosis by inhibiting the activity of HMG-CoA reductase, are mevinolin (U.S. Pat. No. 4,231,938), pravastatin sodium (U.S. Pat. No. 4,346,227) and simvastatin (U.S. Pat. No. 4,444,784) which are fungal metabolites or chemical derivatives thereof More recently a wide variety of synthetic inhibitors of HMG-CoA reductase have been disclosed including certain pyrimidyldihydroxyheptenoic acid derivatives (such as those disclosed in EP 521,471).

We have surprisingly found that a particular pyrimidyldihydroxyheptenoic acid derivative is a potent inhibitor of HMG-CoA reductase, which plays a major role in the synthesis of cholesterol, and thus it may suppress the biosynthesis of cholesterol. This compound is therefore potentially useful in the treatment of hypercholesterolemia, hyperlipoproteinemia and atherosclerosis and other disease conditions mediated by HMG-CoA reductase.

The present invention relates to the compound of formula (I):

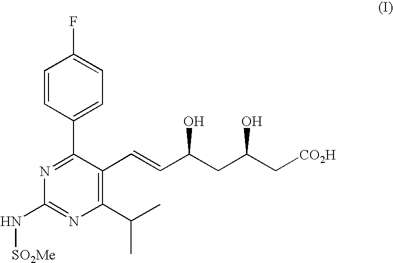

or a pharmaceutically acceptable salt or an in vivo hydrolysable ester thereof.

This compound of formula (I) is conveniently called: (E)-7-[4-(4-fluorophenyl)-6-isopropyl-2-mesylaminopyrimidin-5-yl]-(3R,5S)-dihydroxhept-6-enoic acid.

Suitable pharmaceutically acceptable salts include base salts such as an alkali metal salt for example sodium; an alkaline earth metal salt for example calcium or magnesium; an ammonium salt; or an organic amine salt such as an alkylamine for example methylamine, ethylamine, diethanolamine, tris(hydroxymethyl)methylamine, benzylamine, 4-methoxybenzylamine, triethylamine, morpholine, N-methylpiperidine, N-ethylpiperidine, procaine, dibenzylamine, N,N-dibenzylethylamine or amino acids for example lysine. There may be more than one cation or anion depending on the number of charged functions and the valency of the cations or anions. Preferred pharmaceutically acceptable salts are the sodium salt and the calcium salt. In one aspect of the invention the preferred salt is the sodium salt. In another aspect of the invention the preferred salt is the calcium salt.

The compound of formula (I) may be administered in the form of an in vivo hydrolysable ester. This is an ester which is broken down in the human or animal body to give the compound of the formula (I). Examples of in vivo hydrolysable esters of the compound of the formula (I) are, for example, C₁₋₆alloxymethyl esters for example methoxymethyl; C₁₋₆alkcanoyloxymethyl esters for example pivaloyloxymethyl; phthalidyl esters; C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl; phosphate esters; α-acyloxyallcyl ethers for example acetoxymethoxy and 2,2-dimethylpropionyloxy-methoxy; alkanoyl esters for example benzoyl, phenylacetyl and substituted benzoyl and phenylacetyl; alkoxycarbonyl (to give alkyl carbonate esters); dialkylcarbamoyl and N-(dialkylaninoethyl)-N-alkylcarbamoyl (to give carbamates); dialkylaminoacetyl and carboxyacetyl. Examples of substituents on benzoyl include morpholino and piperazino linked from a ring nitrogen atom via a methylene group to the 3- or 4-position of the benzoyl ring. For the avoidance of doubt, an in vivo hydrolysable ester of the compound of formula (I) also includes the compound of formula (I) in a lactonised (i.e. cyclic ester) form.

A preferred aspect of the invention relates to the compound of formula (I) and pharmaceutically acceptable salts thereof.

It is also to be understood that the compound of formula (I) may exist in solvated as well as unsolvated forms such as, for example, hydrated forms. It is to be understood that the invention encompasses all such solvated forms.

In another aspect the present invention provides a process for preparing the compound of formula (I) or a pharmaceutically acceptable salt thereof which process comprises:

i) deprotecting a compound of formula (II):

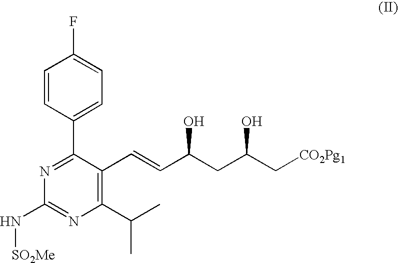

wherein Pg₁ is an acid protecting group; or

ii) deprotecting a compound of formula (III):

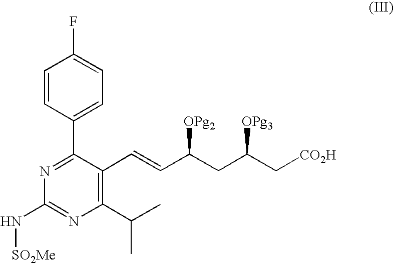

wherein Pg₂ and Pg₃ are alcohol protecting groups or Pg₂ and Pg₃ together form a cyclic alcohol protecting group;

and thereafter if required:

forming a pharmaceutically acceptable salt or an in vivo hydrolysable ester thereof.

Specific reaction conditions for the above reactions are as follows:

Process i) A suitable value for Pg₁ is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid or treatment with an alkali, for example sodium hydroxide, or for example a benzyl group which may be removed, for example, by hydrogenation over a catalyst such as palladium-on-carbon.

Compounds of formula (II) may be prepared according to the following scheme.

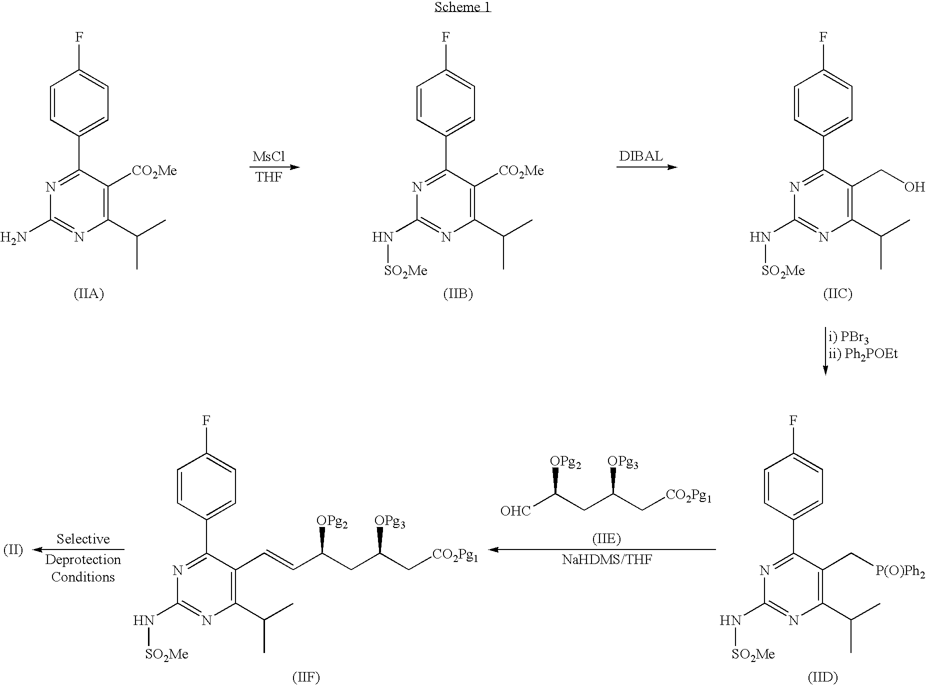

The selective deprotection conditions are those in which Pg₂ and Pg₃ are removed, but not Pg₁.

Compounds of formula (IIA) and (IIE) are known compounds or they are prepared from known compounds by standard conditions known in the art.

Process ii) Suitable values for Pg₂ and Pg₃ are, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon. Where Pg₂ and Pg₃ together form a cyclic alcohol protecting group a suitable value is for example a dimethyl methylene group. This may be removed by treatment with a suitable acid, for example hydrochloric acid.

Compounds of formula (III) may be prepared according to Scheme 1, but wherein the selective deprotection conditions are chosen such that Pg₁ is removed and Pg₂ and Pg₃ remain in place.

In order to use the compound of formula (I) or a pharmaceutically acceptable salt thereof for the therapeutic treatment (including prophylactic treatment) of mammals including humans, it is normally formulated in accordance with standard pharmaceutical practice as a pharmaceutical composition.

The pharmaceutical compositions of the compound of this invention may be administered in standard manner for the disease condition that it is desired to treat, for example by oral, topical, parenteral, buccal, nasal, vaginal or rectal administration or by inhalation. For these purposes the compound may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is oral.

In addition to the compound the pharmaceutical composition of this invention may also contain, or be co-administered (simultaneously or sequentially) with, one or more pharmacological agents of value in treating one or more disease conditions referred to hereinabove.

The pharmaceutical compositions of this invention will normally be administered to humans so that, for example, a daily dose of 0.5 to 200 mg (and preferably of 0.1 to 100 mg for oral administration) is received. This daily dose may be given in divided doses as necessary, the precise amount of the compound received and the route of administration depending on the weight, age and sex of the patient being treated and on the particular disease condition being treated according to principles known in the art.

According to a further aspect of the invention there is provided a pharmaceutical composition which comprises the compound of formula (I) as defined hereinbefore or a pharmaceutically acceptable salt thereof, in association with a pharmaceutically acceptable excipient or carrier.

According to a further aspect of the present invention there is provided the compound of formula (I) or a pharmaceutically acceptable salt thereof, as defined hereinbefore for use in a method of treatment of the human or animal body by therapy.

A further feature of the present invention is the compound of formula (I) or a pharmaceutically acceptable salt thereof, for use as a medicament.

Conveniently this is the compound of formula (I), or a pharmaceutically acceptable salt thereof, for use as a medicament to inhibit HMG-CoA reductase, in a warm-blooded animal such as a human being.

Thus according to a further aspect of the invention there is provided the use of the compound of the formula (I), or a pharmaceutically acceptable salt thereof, in the manufacture of a medicament for use in the inhibition of HMG-CoA reductase in a warm-blooded animal such as a human being.

According to a further feature of the invention there is provided a method of inhibiting HMG-CoA reductase in a warm-blooded animal, such as a human being, in need of such treatment which comprises administering to said animal an effective amount of the compound of formula (I) or a pharmaceutically acceptable salt thereof.

According to a further feature of the invention there is provided the compound of formula (I) or a pharmaceutically acceptable salt thereof in isolated form.

According to a further aspect of the invention there is provided a pharmaceutical composition which comprises the compound formula (I) or a pharmaceutically acceptable salt thereof in isolated form, in association with a pharmaceutically acceptable excipient or carrier.

According to a further aspect of the present invention there is provided the compound of formula (I) or a pharmaceutically acceptable salt thereof in isolated form, for use in a method of treatment of the human or animal body by therapy.

A further feature of the present invention is the compound of formula (I) or a pharmaceutically acceptable salt thereof in isolated form, for use as a medicament.

Conveniently this is the compound of formula (I) or a pharmaceutically acceptable salt thereof in isolated form, for use as a medicament to inhibit HMG-CoA reductase in a warm-blooded animal such as a human being.

Thus according to a further aspect of the invention there is provided the use of the compound of formula (I) or a pharmaceutically acceptable salt thereof in isolated form, in the manufacture of a medicament for use in the inhibition of HMG-CoA reductase in a warm-blooded animal such as a human being.

According to a further feature of the invention there is provided a method of inhibiting HMG-CoA reductase in a warm-blooded animal, such as a human being, in need of such treatment which comprises administering to said animal an effective amount of the compound of formula (I) in isolated form or a pharmaceutically acceptable salt thereof.

Particularly the inhibition of HMG-CoA reductase refers to the treatment of hypercholesterolemia, hyperlipoproteinemia or atherosclerosis.

The term “isolated form” as used herein means that the compound is produced and purified synthetically.

The following Biological Test Methods serve to illustrate the present invention.

Test (a)—Rat Liver Microsomes

Liver microsomes were prepared from normal fed rats by a standard method.

The assay method was a modification of the method described by Kuroda & Endo (Biochimica et Biophysica Acta 486 (1977) 70-81).

The reaction mixture contained in a total volume of 50 μl: 100 mM potassium phosphate buffer pH 7.4, 100 μM DL-[3-¹⁴C]-HMGCoA, 5 mM NADPH, 10 mM EDTA, 10 mM DTT, and 50 μg microsomal protein. The compound of formula (I) at various concentrations was added in 5 μl of 50% DMSO. The reaction was started by addition of the microsomes and the mixture incubated at 37° C. for 30 minutes. After termination of the reaction by addition of 10 μl 2M HCl the mixture was incubated at 37° C. for 15 min to lactonize the product then applied to a silica gel TLC plate (Whatnan LKSD). Chromatograms were developed in toluene: acetone 1:1 and the spots corresponding to products visualized with iodine vapour. Spots corresponding to mevalonolactone were scraped and radioactivity measured in a liquid scintillation counter. Seven-point dose response curves were constructed and the data analysed to determine IC₅₀ values using a curve fitting programme (Origin, Microcal Software Inc).

Test (b)—Human HMGCoA Reductase Catalytic Domain

A soluble catalytic fragment of human EMGCoA reductase containing residues 419-888 was expressed in E coli and purified by affinity chromatography.

The assay method was a modification of the method described by Louis-Flamberg et al (Biochemistry 29 (1990) 4115-4120).

The reaction mixture contained in a total volume of 1 ml: 100 mM MOPS buffer pH 7.2, 200 mM NaCl, 100 mM KCl, 1 mM EDTA, 10 mM DTT, 100 μM HMGCoA, 250 μM NADPH, and 5 nM enzyme. The compound of formula (I) at various concentrations was added in 100 μl of 50% DMSO. The reaction was started by addition of the enzyme and the mixture incubated at 25° C. for up to 20 minutes. HMGCoA reductase activity was assayed continuously by monitoring the decrease in absorbance at 340 nm due to NADPH oxidation in a spectrophotometer (Perkin-Elmer Lambda Bio 40). Data was analysed according to the method of Morrison & Walsh (Advanc. Enzymol.61 (1988) 201-301) in order to determine IC₅₀ values for both initial and steady state rates, using non-linear least squares fitting with Grafit 4.0 software (Erithacus Software 1998).

### EXAMPLE

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mmHg) with abath temperature of up to 60° C.;

(iii) chromatography means flash chromatography on silica gel (Merck Keiselgel ART 9385); thin layer chromatography (TLC) was carried out on silica gel plates; where a “silica Mega Bond Eluf” column is referred to, this means a column containing 10 g of silica of 40 micron particle size, the silica being contained in a 60 ml disposable syringe and supported by a porous disc, obtained from Varian, Harbor City, Calif., USA under the name “Mega Bond Elut SI”; where a “Bond Elut” column is referred to, this means a column containing 10 g of C-18 silica, the silica being contained in a 60 ml disposable syringe and supported by a porous disc of 54 Å pore size, obtained from International Sorbent Technology under the name “ISOLUTE”; “ISOLUTE” is a registered trade mark;

(iv) in general, the course of reactions was followed by TLC and reaction times are given for illustration only;

(v) yields are given for illustration only and are not necessarily those which can be obtained by diligent process development; preparations were repeated if more material was required;

(vi) when given, NMR data is in the form of delta values for major diagnostic protons, given in parts per million (ppm) relative to tetramethylsilane (TIMS) as an internal standard, determined at 300 MHz; s, singlet; d, doublet; dd, double doublet; t, triplet; m, multiplet; br, broad;

(vii) mass spectra were obtained using a caesium fast ion bombardment ionisation source in positive ion mode at 10 keV in a 3-nitrobenzylalcohol matrix; values for m/z are given; generally, only ions which indicate the parent mass are reported; and

(viii) the following abbreviation has been used:

TF tetahydrofuran.

The compound of formula (I) may be prepared according to the following scheme:

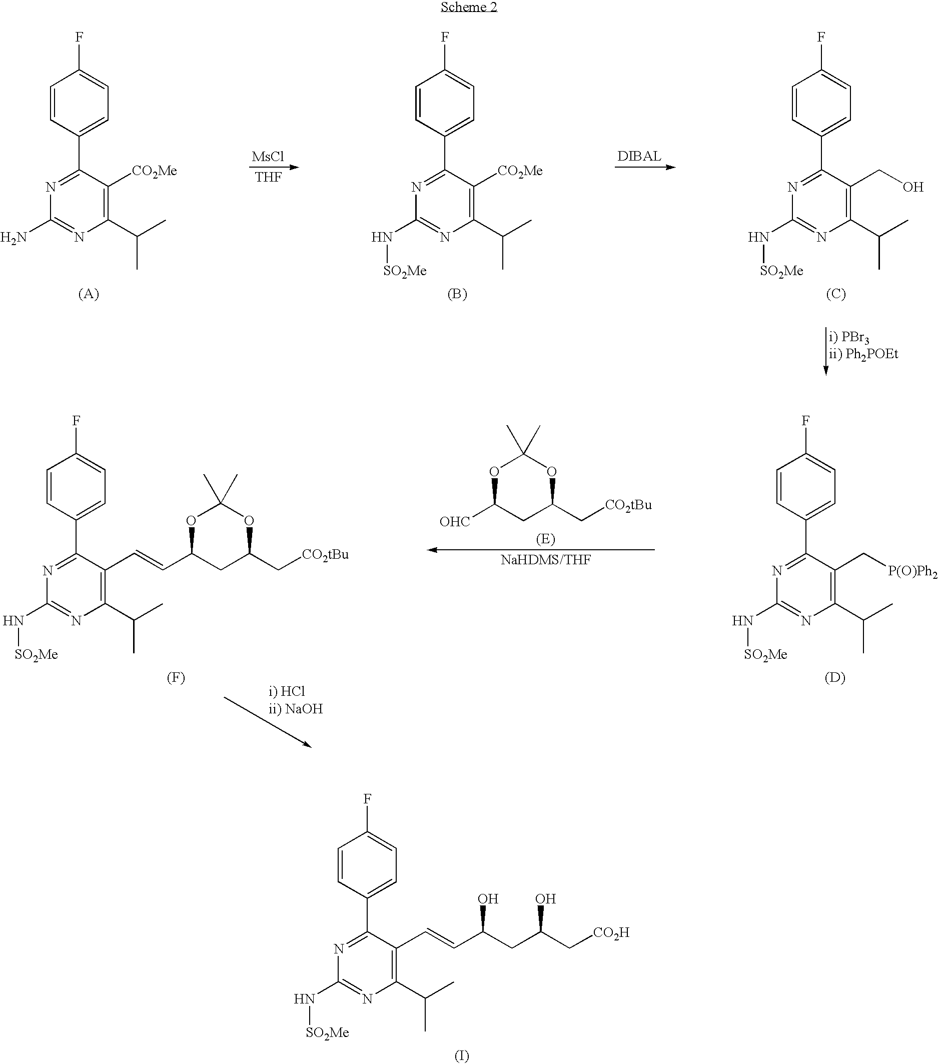

### Example 1

Compound of Formula (I)

Compound F (method 4; 200 mg) was dissolved in acetonitrile (5 ml) under nitrogen. The reaction mixture was heated to 35° C. and then hydrochloric acid (0.535 ml, 1M solution) was added dropwise. The reaction mixture was heated at 35° C. for 3 hours and then cooled to 25° C. over 30 mins. Sodium hydroxide (1.07 ml, 1M solution) was added dropwise until the solution became pH 12. The solution was stirred at room temperature and then additional sodium hydroxide (0.3 ml, 1M) was added and the pH increased to 13.25. The mixture was stirred at room temperature for a further 30 mins and then stored in a refrigerator overnight. The aqueous phase was freeze dried for 6 hours to yield a solid (0.1754 g). The solid was purified by solid phase extraction. The solid was dissolved in an mixture of ethyl acetate and methanol (1:1) and loaded onto a 10 g silica Mega Bond Blut column. The material was eluted with ethyl acetate (50 ml) and ethyl acetate: ethanol; 8:2 (50×5 ml) to yield a colourless oil with traces of solid (77 mg). A C-18 Bond Elut cartridge (10 g) was preconditioned with acetonitrile (30 ml) and formic acid (0.01M) (30 ml) and the oil was purified on the Bond Blut column eluting with acetonitrile: formic acid (0.01M); 2:8 increasing to 4:6 to yield an oil (54.4 mg). NMR 1.2 (d, 6H), 1.35 (m, 1H), 1.5 (m, 1H), 2.2 (dd, 11, 2.3 (dd, 1H), 3.4 (s+m, 4H), 3.85 (m, 1H), 4.15 (m, 1H), 4.85 (brs, 1H), 5.6 (dd, 1H), 6.45 (dd, 1H), 7.25 (t, 2H), 7.65 (m, 21); m/z 468 (MH³⁰).

Preparation of Starting Materials

The starting materials for the Example above are either commercially available or are readily prepared by standard methods from known materials. For example the following reactions are illustrations but not limitations of the preparation of some of the starting materials used in the above reactions.

Method 1

Compound B

Sodium t-pentoxide (0.4420 g) and dimethoxyethane (5 ml) were added to Compound A (JP 06256318; 0.5007 g) under argon and the mixture was stirred at room temperature for 30 mins, before cooling to −10° C. Methane sulphonyl chloride (0.22 ml) was added dropwise and the reaction was stirred at −10° C. for a further 20 mins before water was added (3.5 ml). The organic layer was separated and evaporated to dryness to yield a pale yellow residue. This was purified by column chromatography eluting with petrol: ethyl acetate; 90:10 increasing to 85:15 to give a white solid (0.5217 g, 82%). NMR (CDCl₃): 1.25 (d, 61, 3.10 (m, 1H), 3.42 (s, 3H), 3.65 (s, 3H), 7.1 (t, 2H), 7.5 firs, 1H), 7.6 (m, 2H); m/z 368 (MH³⁰).

Method 2

Compound C

Toluene (0.6 ml) was added to Compound B (Method 1; 135 mg) under argon and the suspension was cooled to −10° C. Diisobutylaluninium hydride (0.85 ml of a 1.5M solution in toluene) was added and the resulting yellow suspension was stirred at −10° C. for a further hour. Methanol (10 μl) was added and the reaction mixture was allowed to warm to room temperature. In a separate flask, concentrated hydrochloric acid (0.25 ml) and water (0.45 ml) were mixed and heated to 40° C. The reaction mixture was added dropwise to the acid solution and the resulting solution was heated at 40° C. for 20 mins before allowing to cool to room temperature. The organic layer was separated and evaporated to dryness to yield a yellow oil. This was purified by column chromatography eluting with petrol: ethyl acetate; 80:20 increasing to 70:30 to give a colourless oil (102 mg). M/z 340 (MH³⁰).

Method 3

Compound D

Dry dichloromethane (15 ml) was added to Compound C (Method 2; 1.2328 g) under argon and the mixture was stirred for 30 mins. Phosphorous tribromide (173 μl) was added and the mixture was stirred at room temperature for 1 hour. Water (30 ml) was added and the organic layer was separated. The aqueous layer was re-extracted with dichloromethane (15 ml) and the combined organic layers were dried and evaporated to dryness to give a white solid. The white solid was dissolved in dry toluene (28 ml) under argon and the mixture was heated to 60° C. Ethyl diphenylphosphinite (1.18 ml) was added and the mixture was heated at 60° C. for 3 hours during which tine a white solid precipitated out of the solution. The reaction mixture was stored in a refrigerator for 48 hours and then filtered. The resulting solid was washed with toluene and dried (1.625 g, 85%). NMR (DMSO-d₆): 1.1 (d, 6H), 3.2 (m, 1H), 3.35 (s, 3H), 3.95 (d, 2H), 7.0-7.3 (m, 6H), 7.4 (m, 4H), 7.55 (m, 6H); m/z 524 (MW).

Method 4

Compound F

Compound D (Method 3; 1.5033 g) was dissolved in dry TBF (22 ml) under argon and the solution was cooled to −60° C. Sodium hexamethyldisilazane (5.7 ml of a 1M solution in THF) was added dropwise and the reaction was stirred at −60° C. for 20 mins. Compound E (EP 319847, 5.72 ml of 16.4% w/w solution in toluene) was added dropwise and the reaction was stirred at −60° C. for 15 mins and then allowed to warm up gradually to 10° C. Water (1.5 ml) was added followed by glacial acetic acid (0.5 ml) and the temperature was increased to 25° C. The mixture was stirred at ambient temperature for 15 mins and then the TEF was removed by evaporation. Additional water (5 ml) and toluene (2 ml) were added and the organic layer was separated and washed with saturated sodium hydrogen carbonate solution (5 ml) and water (5 ml). The aqueous layers were re-extracted with ethyl acetate (5 ml) and the combined organic layers were evaporated to dryness to yield an oil which solidified on standing. The residue was pre absorbed onto silica by dissolving in methanol and then purified by column chromatography eluting with isohexane:ethyl acetate; 8:2 to yield a yellow oil (1.088 g, 67.5%). NMR (DMSO-dk): 1.2 (d, 6H), 1.49 (s, 91), 2.21 (dd, 2H), 2.39 (dd, 21), 3.3 (s, 3H+m, 1H), 3.35 (s, 6H), 4.22 (m, 1H), 4.52 (m, 1H), 5.41 (dd, 1H), 6.52 (d, 1H), 7.25 (t, 2H), 7.6 (m, 2H), 11.25 (brs, 1H); m/z 564 (MH³⁰).

What we claim is:

1. The compound of formula (I) 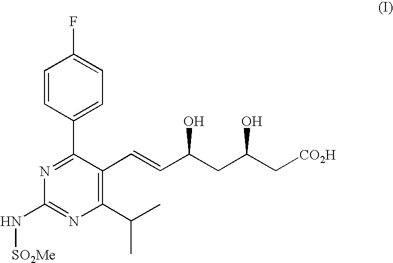
or a pharmaceutically acceptable salt or an in vivo hydrolysable ester thereof.

2. The compound as claimed in claim 1 in the form of an alkali metal salt, an alkaline earth metal salt, an ammonium salt or an organic amine salt.

3. The compound as claimed in claim 1 or 2 in the form of a sodium or calcium salt.

4. A process for preparing the compound of formula (I) as claimed in claim 1, or a pharmaceutically acceptable salt thereof or an in vivo hydrolysable ester thereof, which process comprises:
i) deprotecting a compound of formula (II) 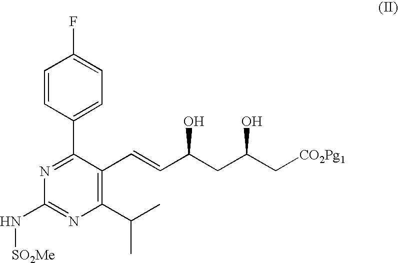
wherein Pg₁ is an acid protecting group; or
ii) deprotecting a compound of formula (III) 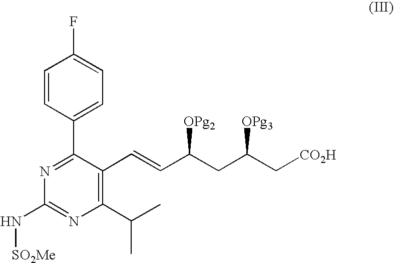
wherein Pg₂ and Pg₃ are alcohol protecting groups or Pg₂ and Pg₃ together form a cyclic alcohol protecting group;
and thereafter if required:
forming a pharmaceutically acceptable salt or an in vivo hydrolysable ester thereof.

5. A pharmaceutical composition which comprises the compound of formula (1) as defined in claim 1, or a pharmaceutically acceptable salt thereof or an in vivo hydrolysable ester thereof, in association with a pharmaceutically acceptable excipient or carrier.

6. The compound of formula I as defined in claim 1, or a pharmaceutically acceptable salt thereof or an in vivo hydrolysable ester thereof, for use in a method of treatment of the human or animal body by therapy.

7. The use of the compound of formula I as defined in claim 1, or a pharmaceutically acceptable salt thereof or an in vivo hydrolysable ester thereof, in the manufacture of a medicament for use in the inhibition of HMG-CoA reductase in a warm-blooded animal.

8. A compound of the formula (II) 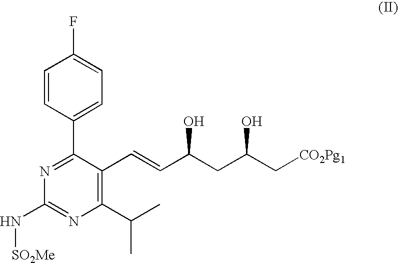
wherein Pg₁ is an acid protecting group.

9. A compound of the formula (III) 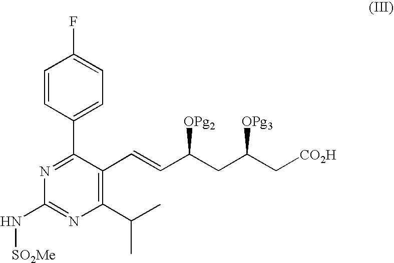
wherein Pg₂ and Pg₃ are alcohol protecting groups or Pg₂ and Pg₃ together form a cyclic alcohol protecting group.
